(19) United States
(12) Patent Application Publication
(10) Pub. No.: US 2005/0272990 A1
(43) Pub. Date: Dec. 8, 2005

(54) Method and apparatus for non-invasively monitoring concentrations of glucose or other target substances

(75) Inventors: Arie Ariav, Doar-Na Hof Ashkelon (IL); Vladimir Ravitch, Ashkelon (IL); Benjamin J. Barish, Tel Aviv (IL)

Correspondence Address: Martin Moynihan;c/o Anthony Castorina, Suite 207, 2001 Jefferson Davis Highway, Arlington, VA, 22202, US

(73) Assignee: Nexense Ltd., Yavne (IL)

(21) Appl. No.: 11/125,189

(22) Filed: May 10, 2005

Related U.S. Application Data
(63) Continuation-in-part of application No. 10/844,398, filed on May 13, 2004.

(30) Foreign Application Priority Data
Feb. 8, 2005 (IL) 166760

(51) Int. Cl. A61B 5/00

(52) U.S. Cl. 600/365; 600/309

(57) ABSTRACT

A method and apparatus for non-invasively measuring concentration of a target substance such as glucose within a body by: activating a pulse source to apply to the body a series of pulses of energy highly absorbable by the target substance to generate, by the photoacoustic effect, a series of acoustic waves propagated through an acoustic channel in the body; detecting the acoustic waves to produce an electrical signal having a frequency corresponding to the frequency of the acoustic waves generated by the photoacoustic effect; controlling the pulse source to change the frequency at which the energy pulses are applied to the body such that the detector detects a whole integer number of wavelengths in the acoustic channel irrespective of variations in the target substance concentration within the body; and utilizing a measurement of the frequency, or change in frequency, of the pulses to produce a measurement of the concentration, or change in concentration, of the target substance.

### RELATED APPLICATIONS

The present application is a continuation-in-part of U.S. patent application Ser. No. 10/844,398, filed May 13, 2004, and also includes subject matter of Israel Patent Application 166,760 filed Feb. 8, 2005, the priority date of which is also claimed.

### FIELD AND BACKGROUND OF THE INVENTION

The present invention relates to a method and apparatus for non-invasively monitoring the concentration of a target substance in a body. The invention is particularly useful for measuring the concentration, or changes in the concentration, of glucose within the blood of a person, and is therefore described below with respect to that application, but it will be appreciated that the invention could advantageously be used in many other applications.

Frequent monitoring of blood glucose level is critical for those suffering from diabetes. Currently, glucose measurements are generally performed by the individual, by pricking a finger tip and applying a drop of blood to a test strip composed of chemicals sensitive to the glucose in the blood sample. However, this method is very painful and usually inconvenient, particularly when done many times (e.g., 4-7 times) per day as recommended.

It is presently estimated that over 18 million people in the USA suffer from diabetes, and that this number will dramatically increase, to about 24 million in 2010. Considerable research and development has been conducted along many different avenues in an attempt to develop an effective non-invasive glucose monitoring device, as shown by the many technical articles that have been published on this subject and the many patents that have issued. Nevertheless, despite this dramatically increasing need for a method for monitoring blood glucose levels in a non-invasive, painless and convenient manner, and despite the considerable research and development efforts that have been devoted to developing such a device, no such device is yet commercially available, insofar as we are aware, having the accuracy, reliability and repeatability needed for general use.

While this problem is particularly acute with respect to monitoring blood glucose levels, the problem is also present in monitoring the concentration of other constituents of blood, such as cholesterol, or the constituents of urine, or of other biological fluids, industrial fluids, other bodies, etc.

### OBJECTS AND BRIEF SUMMARY OF THE PRESENT INVENTION

An object of the present invention is to provide a new, non-invasive method of measuring the concentration, or change in concentration, of a target substance within a body. Another object of the invention is to provide a method particularly useful for measuring the concentration, or change in concentration, of glucose within the blood of a person. A further object is to provide novel apparatus for non-invasively measuring the concentration, or change in the concentration, of a target substance, particularly blood glucose.

According to one aspect of the present invention, there is provided a method of non-invasively measuring the concentration, or change in concentration, of a target substance within a body, comprising the operations: activating a pulse source to apply to the body a series of pulses of energy highly absorbable by the target substance, as compared to other substances, to heat the body and to generate therein, by the photoacoustic effect, a series of acoustic waves propagated through an acoustic channel in the body at a frequency corresponding to that at which the energy pulses are applied to the body; detecting the acoustic waves to produce an electrical signal having a frequency corresponding to the frequency of the acoustic waves generated by the photoacoustic effect, and thereby to the frequency at which the energy pulses are applied to the body; controlling the pulse source to change the frequency at which the energy pulses are applied to the body, and thereby the frequency of the acoustic waves, such that the detector detects a whole integer number of wavelengths in the acoustic channel irrespective of variations in the target substance concentration within the body; and utilizing a measurement of the frequency, or change in frequency, of the pulses to produce a measurement of the concentration, or change in concentration, of the target substance.

The “photoacoustic effect” utilized in the above method is well known and has long been used for non-invasively producing various types of measurements, e.g. temperature, pressure, composition, etc. It has also been proposed for use in non-invasively monitoring blood glucose levels, as described for example in U.S. Pat. Nos. 5,348,002, 5,348,003, 5,941,821, 6,833,540, and 6,846,288 Insofar as we are aware, however, a method utilizing this effect has not yet been implemented in a commercially-available device or in a device which has obtained FDA approval.

As will be described more particularly below, the present invention utilizes the “photoacoustic effect”, together with a method, herein referred to as the Frequency-Change by Wavelength-Control (or FCWC) method described in U.S. Pat. No. 6,621,278 (Israel Patent 129651), assigned to the same assignee as the present application, for producing a glucose monitoring device capable of achieving high reliability without a need for frequent recalibration as compared to other known methods.

The FCWC method, as described in U.S. Pat. No. 6,621,278, is capable of measuring with extremely high precision the transit time of an energy wave (compressional or electromagnetic) moving through a transmission channel. The method thus enables measuring, with extremely high precision, virtually any parameter or condition having a known relation to, or influence on, the transit time of movement of an energy wave through a medium. Briefly, this is done by: (a) transmitting a cyclically-repeating energy wave through a transmission channel in the medium; (b) changing the frequency of the transmission according to changes in the monitored condition, while maintaining the number of wavelengths in the transmission channel as a whole integer; and (c) utilizing the changes in frequency of the transmission to provide a measurement of the monitored condition. The change in frequency thus reflects the change in transit time of the energy wave attributed to the monitored condition. This change in transit time may result from a change in the transit velocity, and/or a change in the transit distance of the energy wave through the transmission channel. Further details of the FCWC method are available in U.S. Pat. No. 6,621,278.

When the FCWC method is used in the present invention, the energy wave transmitted through the transmission channel is the acoustic wave generated by the “photoacoustic effect”; and the medium of the channel is the body containing the target substance to be monitored, e.g. glucose in a patient's blood.

Embodiments of the present invention are described below which utilize the FCWC (Frequency-Change by Wavelength-Control) method described in the above-cited U.S. Pat. No. 6,621,278, to produce a precise measurement of the transit time of an acoustic wave through a transmission channel, and thereby of the concentration of the target substance being monitored to the extent that it changes this transit time by a change in the transit velocity and/or the transit distance. This aspect of the present invention utilizes the selective absorption of energy by the target substance, and particularly the “photoacoustic effect”, for generating the acoustic waves used in the FCWC method. Accordingly, the present invention enables changes in glucose concentration to be measured with a high degree of accuracy, reliability and repeatability.

The invention, however, can also be implemented by using the FCWC method without the “photoacoustic effect”, in order to measure the concentration of the glucose (or other target substance) according to the heat generated by the target substance, since such generated heat also changes the transit time of an acoustic wave through an acoustic channel.

According to another aspect of the present invention, therefore, there is a provided a method of non-invasively measuring the concentration of a target substance within a body, comprising: transmitting acoustic waves through an acoustic wave transmission channel in the body to a detector at the opposite end of the acoustic wave transmission channel; applying to the body in the acoustic wave transmission channel energy highly absorbable by the target substance, as compared to other substances, to heat the portion of the body within the acoustic wave transmission channel according to the concentration of the target substance in the body; detecting the acoustic waves in the transmission channel to output an electrical signal having a frequency corresponding the frequency of the acoustic waves transmitted through the channel by the acoustic wave transmitter; controlling the acoustic wave transmitter to change the frequency thereof such that the detector detects a whole integer number of wavelengths irrespective of variations in the target substance concentration with the body; and utilizing the frequency of the detector output signal to produce a measurement of the target substance concentration. The magnitude of the detector output signal may also be used in producing the measurement of the target substance concentration.

An advantage of this aspect of the present invention is that it enables the FCWC method to be used in two independent manners for measuring the concentration of the target substance. Thus, it uses the selective heating by the target substance to produce, by the “photoacoustic effect”, the acoustic waves used in the FCWC method. It also enables the increase in temperature produced by the selective heating to be precisely measured by the FCWC method to provide a measurement of the glucose concentration. In both cases, the FCWC method enables precisely measuring the change in transit time of the acoustic wave, and thereby any condition such as the change in temperature and/or composition, affecting the transit velocity of the acoustic wave. Thus, both techniques can be used in any particular monitoring operation, in order to improve the accuracy and reliability of the final result by executing one technique to extract data from the monitored site useful to determine concentration by the other techniques, or to corroborate the results produced by the other technique.

The present invention also enables a number of acoustic channels to be established through the monitored region for extracting therefrom various types of information which can be used to reduce the extraneous influences, and thereby to provide a more accurate measurement of the concentration of the target substance within the body.

According to another aspect of the present invention, therefore, there is provided a method of non-invasively measuring the concentration, or change in concentration, of a target substance within a body, comprising: transmitting acoustic waves through at least two separate acoustic channels in the body; applying to one of the channels energy which is selectively absorbable by the target substance to thereby heat the respective channel according to the concentration of the target substance therein; and measuring the difference in temperature between that in the one channel with respect to that in the other channel, to thereby provide a measure of the concentration, or change in concentration, of the target substance in the body.

According to still further aspects, the invention also provides apparatus for non-invasively measuring the concentration, or change in the concentration, of a target substance within a body according to the above methods.

In the described preferred embodiments, the pulse source is a laser having a wavelength selectively absorbable by the target substance; and the target substance is a constituent of the blood of a person, particularly the glucose in the person's blood. It will be appreciated, however, that the invention can use other pulse sources and can be used for determining the concentration, or change in concentration, of other target substances within other bodies.

Further features an advantages of the invention will be apparent from the description below.

### BRIEF DESCRIPTION OF THE DRAWINGS

The invention is described below, for purposes of example only, with respect to the accompanying drawings, wherein:

FIG. 1 is a block diagram illustrating one form of apparatus for non-invasively monitoring the concentration, or change in concentration, of a target substance, particularly glucose within blood, in accordance with the present invention;

FIG. 2 is a block diagram illustrating the FCWC (Frequency-Change by Wavelength-Control) system of the above-cited U.S. Pat. No. 6,621,278 as used in the apparatus of FIG. 1;

FIG. 3 illustrates another apparatus constructed in accordance with the present invention for non-invasively monitoring glucose concentration;

FIG. 4 illustrates a modification in the apparatus of FIG. 3;

FIG. 5 illustrates yet another apparatus constructed in accordance with the present invention for non-invasively monitoring glucose concentration in blood; and

FIG. 6 illustrates the use of two monitoring sites for non-invasively monitoring glucose concentration.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

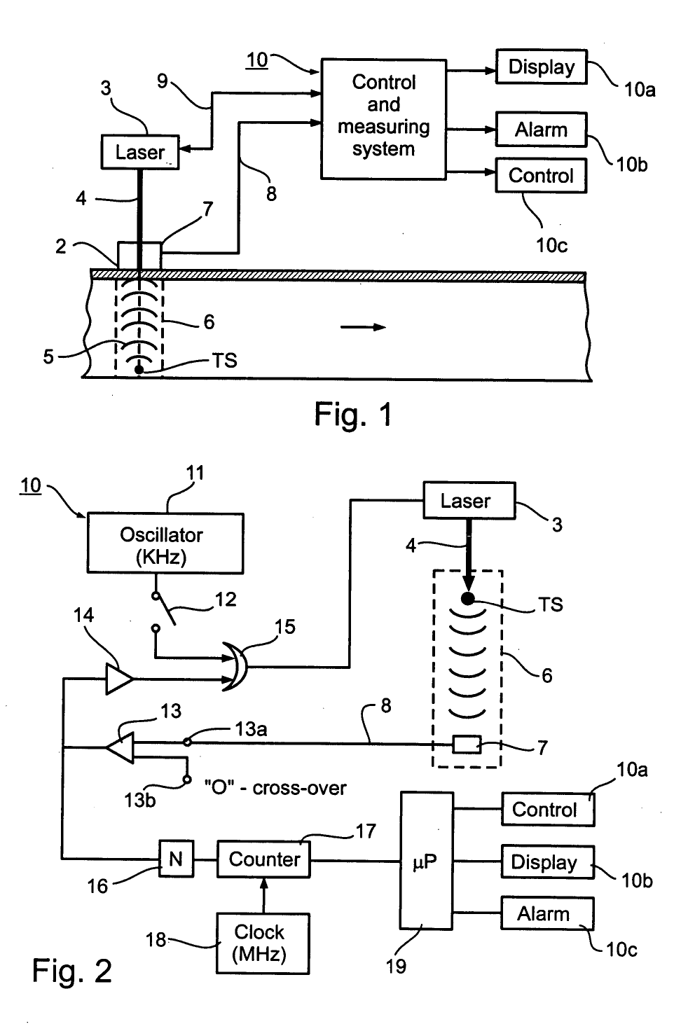

The Embodiment of FIG. 1

The apparatus illustrated in FIG. 1 is for non-invasively monitoring changes in the concentrate of a target substance TS in the blood flowing through a monitored site 2 of a person. As indicated earlier, the method described is particularly useful for monitoring changes in the concentration of glucose in blood. Therefore the target substance TS is hereinafter referred to as glucose, but it will be appreciated that the invention could also be used for monitoring other target substances in other bodies, such as other constituents of blood, or constituents of urine, constituents of other biological fluids or other types of fluids, e.g., industrial fluids, or constituents of other bodies, i.e., solids and gases as well as liquids.

The apparatus illustrated in FIG. 1 includes a laser 3 which applies laser pulses via an optical fiber 4 to a selected region of the monitored site 2. Laser 3 may include a single laser, or a combination of lasers, having a wavelength or combination of wavelengths selectively absorbable by the glucose TS within the blood flowing through monitored site 2, as compared to other substances in the region exposed to the laser energy. As a result, the absorption of the laser energy by the glucose is effective to heat the respective region according to the glucose concentration in the blood. This absorption of the laser energy by the glucose generates, by the “photoacoustic effect”, a series of acoustic waves, shown at 5 in FIG. 1, which are propagated through an acoustic channel 6 at a frequency corresponding to that at which the laser is pulsed. The acoustic waves so generated in channel 6 by the glucose TS are detected by an acoustic detector 7 in contact with the external surface of the skin at the monitored site. For this purpose, acoustic detector 7 may be formed with a central opening to accommodate optical fiber 4 from the laser.

The frequency of activation of laser 3, and thereby the frequency of generation of the acoustic waves 5 by the photoacoustic effect, is controlled by detector 7 via a control lines 8 and 9, and a control and measuring system, generally designated 10. System 10 is constructed as described in the above-cited U.S. Pat. No. 6,62,278 and is illustrated in FIG. 2 of the accompanying drawings. As will be described more particularly below with respect to FIG. 2, such a system controls laser 3 in accordance with the above-described FCWC (Frequency-Change by Wavelength-Control) method, to change the frequency of application of the laser energy pulses, and thereby the frequency of the acoustic waves 5 through channel 6, such that the detector detects a whole integer number of wavelengths in channel 6 irrespective of the concentration of the glucose TS. Thus, the frequency of the laser pulses is changed by, and according to, changes in the concentration of glucose at the monitored site 2, so that the frequency change represents a measure of the glucose concentration change.

As shown in FIG. 1, the measurements produced by system 10 may be outputted to the following output units: a display unit 10a, such as a display in a wrist-worn monitoring device; an alarm unit 10b, such as a sounder or vibrator actuated to alert the person of an alarm condition; and/or a control device 10c, such as an automatic control for an insulin-delivery pump.

The Control and Measuring System of FIG. 2

FIG. 2 more particularly illustrates the control and measuring system 10 of FIG. 1 for controlling the frequency of activation of laser 3, and thereby the generation of the acoustic waves 5 detected by detector 7, for maintaining the number of wavelengths of the acoustic waves as whole integer within channel 6 irrespective of the concentration of the glucose TS.

Initially, laser 3 is activated via line 9 by an oscillator 11 under the control of a switch 12 until the acoustic waves 5 are received by detector 7. Once these waves are received, switch 12 is opened, so that the received waves are thereafter used for controlling the activation of laser 3, and thereby the generation by the photoacoustic effect of the acoustic waves 5.

As shown in FIG. 2, the output of detector 7 is fed via line 8 to input 13a of a comparator 13. Comparator 13 includes a second input 13b connected to a predetermined bias so as to detect a predetermined fiducial or reference point in the received signal. In the example illustrated in FIG. 2, this predetermined fiducial point is the “zero” crossover point of the signal outputted from detector 7; hence, input 13b is at a zero bias. However, other reference points could be used as the fiducial point, such as the maximum peaks, the minimum peaks, or the leading edges of the output signal from detector 7.

The output of comparator 13 is fed to a monostable oscillator 14 which is triggered to produce an amplified output signal at each fiducial point in the output signal from detector 7. The signals from monostable oscillator 14 are fed via an OR-gate 15 to control line 9 controlling the activation of laser 3.

It will thus be seen that laser 3 is activated at a frequency such that the photoacoustic waves 5, generated in channel 6 by the absorption of its energy by the targetted glucose TS, is a while integer. The changes in frequency of activation of laser 3, to maintain the number of waves 5 in channel 6 as a whole integer, thus represent a precise measurement of the changes in transit time of the waves 5 from the targetted glucose TS to the detector 7 resulting from the changes in the concentration of the glucose.

The precise measurement of the transit time of the glucose-generated acoustic waves to the detector 7 thus enables a precise measurement to be made of any parameter or condition affecting that transit time. The transit time depends on the transit velocity and the transit distance. Where the transit distance is known or determinable, the measured transit time will be a measure of the transit velocity, and thereby a measure of any factors, such as the heat generated by the glucose, on the transit velocity. Since the heat generated corresponds to the concentration of the glucose, the measured transit time will thus be a measure of the concentration of the glucose at the monitored site.

In addition to heat, other factors, such as changes in composition other than in the glucose concentration, may also affect the transit time of the acoustic wave through channel 6, but such influences for the large part can be determined beforehand or independently, in order to compensate for their influences on the measurements made.

FIG. 2 also illustrates a circuit for accumulating small changes in frequency, over a large time interval as also described in the above-cited U.S. Pat. No. 6,621,278. Thus, as shown in FIG. 2, the signals outputted from comparator 13, used for controlling the frequency of activation of laser 3, are also fed to a counter 16 to be counted “N” times, and the output is fed to another counter 17 controlled by a clock 18. Counter 17 produces an output to a microprocessor 19 which performs the computations according to the parameter or condition to be measured, in this case the concentration of the targetted glucose TS at the monitored site. Microprocessor 19 produces the outputs to the display unit 10a, alarm unit 10b, and/or control unit 10c.

Further details of the construction, use and other possible applications of the system illustrated in FIG. 2 are set forth in the above-cited U.S. Pat. No. 6,621,278.

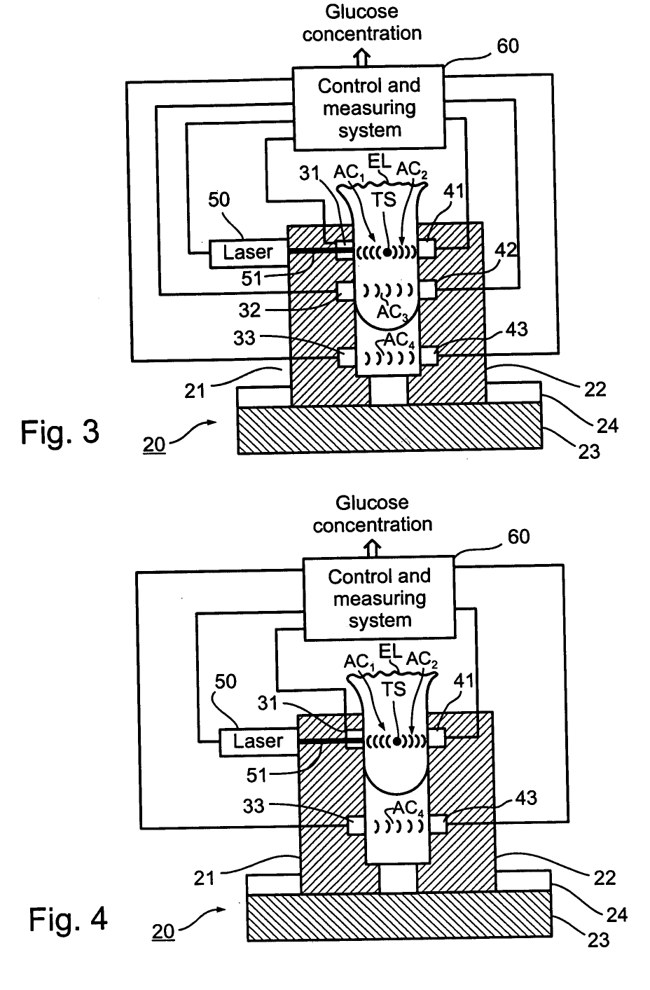

The Embodiment of FIG. 3

FIG. 3 illustrates a further embodiment of the invention also utilizing acoustic waves generated by the photoacoustic effect. This embodiment provides a number of acoustic channels each of which may be used for extracting various types of information from the monitored site to enable a more accurate determination to be made of the concentration of the targetted glucose. Whereas in the embodiment illustrated in FIGS. 1 and 2 the monitored site requires access only from one side, (e.g., such as the person's wrist or fingertip), the embodiment illustrated in FIG. 3 requires a monitored site providing access from both sides, such as an ear lobe, finger web, or the like. For purposes of example, the person's ear lobe is used as the monitored site in FIG. 3.

The apparatus illustrated in FIG. 3 includes a sensor assembly, generally designated 20, for application to the ear lobe EL of the person. Sensor assembly 20 includes two plates 21, 22 slidably mounted on a holder 23, for engaging the opposite surfaces of the ear lobe EL. The two plates 21, 22 are movable within a channel 24 formed in holder 23 so as to maintain the two plates in exact parallel relationship to each other when engaging the opposite surfaces of the ear lobe.

The inner surface of each of plate 21, 22 carries three vertically-spaced acoustic transducers 31, 32, 33, and 41, 42, 43 respectively, aligned with each other when the sensor assembly 20 is mounted to the person's ear lobe. Thus, as shown in FIG. 3, transducers 31 and 41 define a first pair aligned with an intermediate region of the ear lobe; transducers 32 and 42 define a second pair aligned with a lower region of the ear lobe; and transducers 33 and 43 define a third pair aligned with each other in the space below the ear lobe. The distance between the transducers in each of the pairs is equal and is either known or determinable, as will be described below.

Plate 21 of sensor assembly 20 also carries a laser 50 on its outer surface in alignment with acoustic transducer 31 on the inner surface of the plate. Transducer 31 is formed with a central opening to accommodate an optical fiber 51 extending from laser 50 to the inner face of plate 21 to be in contact with the outer surface of ear lobe EL.

Laser 50, and the three pairs of acoustic transducers 31-33 and 41-43, respectively, are connected to a control and measuring system 60. System 60 corresponds to the control and measuring system 10 illustrated in FIGS. 1 and 2, but modified to accommodate three acoustic channels, rather than the one illustrated in FIGS. 1 and 2.

FIG. 3 illustrates target substance TS (e.g., glucose), whose concentration is to be monitored, in alignment with optical fiber 51 from laser 50, acoustic transducer 31 on one side, and acoustic transducer 41 on the opposite site. Thus, when laser 50 is activated as described above with respect to FIGS. 1 and 2, the glucose TS generates a series of acoustic waves by the photoacoustic effect. The so-produced acoustic waves propagate outwardly in all directions, including the direction towards transducer 31, and the opposite direction towards transducer 41. Thus, a first acoustic wave channel AC₁ is established between the glucose TS serving as the generator or transmitter of the acoustic waves, and transducer 31 serving as the detector of the acoustic waves. Similarly, a second acoustic channel AC₂ is established between the glucose and detector 41 on the opposite side.

The illustrated sensor assembly 20 defines two further acoustic channels, namely a third channel AC₃ within a lower part of the ear lobe between the two transducers 32, 42; and a fourth channel AC₄ in the space (air) between the two transducers 33 and 43 below the ear lobe. It will also be seen that the length of acoustic channel AC₃ is equal to that of AC₄ and is also equal to the sum of the two acoustic channels AC₁ and AC₂.

Each of the above four acoustic channels AC₁-AC₄ is controlled by the control and measuring system 60 in the same manner as system 10 described above with respect to FIGS. 1 and 2, to precisely determine the transit time of the acoustic waves in the respective channel. As described above, the so-measured transit time is a measure of the transit velocity through the respective channel AC₁-AC₄, and therefore of any condition influencing the transit velocity. The transit time also depends on the transit distance in the respective channel, but as indicated above, the transit distance is either previously known according to the settings of the two plates 21, 22, or is precisely determinable as will be described below.

Information Extractable from Channel AC,

As indicated above, the transmitter in acoustic channel AC₁ is the targetted glucose TS generating the photoacoustic waves which are detected by acoustic detector 31. Since the transit time of a laser beam from laser 50 to the target substance TS is negligible when compared to the transit time of acoustic waves generated by the glucose, the frequency of activation of laser 50 would be controlled by detector 31, via the control and measuring system 60, in the manner as described above with respect to FIG. 2, such that detector 31 detects a whole integer number of wavelengths irrespective of variations in the glucose concentration.

The frequency of activation of laser 50, and therefore the frequency of the output signal from detector 31, is thus a precise measurement of the transit time in channel AC₁. This frequency can be used to provide information as to the transit distance, i.e., the length of channel AC₁ between the target substance TS and its detector 31. It can also be used to provide information as to any conditions influencing the transit velocity of the generated acoustic waves through channel AC₁.

The magnitude of the output signal from detector 31 is also a measure of the concentration of the glucose in the monitored site. Since the magnitude measurement is an analog signal, it is inherently less accurate than the frequency-change digital signal produced by the FCWC method described above with respect to FIG. 2.

Nevertheless, since the magnitude of the output signal at detector 31 represents a measure of the glucose concentration at the transmitter end of channel AC₁, reduced by the transit distance to the detector 31, and by the acoustic impedance of the medium in channel AC₁, it can also provide information useful in determining the glucose concentration at the monitored site. Thus, the transit distance is determinable with high accuracy from the other information extractable from all the channels AC₁-AC₄ as will be described more particularly below. The acoustic impedance within the channel is influenced not only by the composition of the medium (constituted of tissue plus blood, including the targetted glucose constituent), but also by the temperature of the medium of channel AC₁. As more particularly described below, the latter influences are also determinable by the information extractable from the monitored site by the activation of a selected combination of the channels AC₁-AC₄.

Information Extractable from Channel AC₂

The transmitter in acoustic channel AC₂ is also the targetted glucose TS generating the photoacoustic waves, but in this channel such waves are detected by detector 41. This channel would be activated by the control and measuring system 60 as described above, except that in this case, detector 41 (rather than detector 31) controls the activation of laser 50 to produce a whole integer number of wavelengths within channel AC₂ irrespective of variations in the glucose concentration in that channel. It will therefore be seen that, as described above with respect to channel AC₁, the frequency of the output signal from detector 41 would be a measure of the transit time of the acoustic signal in channel AC₂ (and thereby transit distance and the factors influencing transit velocity in channel AC₂); and that the magnitude of the output signal from detector 41 would be a measure of the glucose concentration, diminished by the transit distance and the acoustic impedance of that channel.

Information Extractable from Combined Channel AC₁ plus AC₂

As indicated above, detector 31 may be operated as an acoustic transmitter to generate acoustic waves propagated through both channels AC₁ and AC₂ to the detector 41. In such an operation, the acoustic waves would be generated by transducer 31, rather than by the photoacoustic effect described above; and the length of the respective channel would be the sum of the lengths of channels AC₁ plus AC₂ During this operation, detector 41 would control, via control and measuring system 60, transmitter transducer 31 to maintain a whole integer number of acoustic waves within the combined channel AC₁ plus AC₂.

Accordingly, during this combined-channel mode of operation of the illustrated apparatus, the frequency of the output signal from detector 41 would be a precise measurement of the transit time of the acoustic wave from transmitter 31 to detector 41, and thereby a measure of the transit distance and/or the transit velocity within this combined acoustic channel. The transit distance, during this operation, is the sum of the transit distances of channels AC₁ and AC₂ referred to in the above-described operations for extracting information from these two channels when individually activated. The transit velocity, on the other hand, would depend on the factors, including the nature of the medium (tissue plus blood including its glucose constituent), and the temperature of the medium, influencing the transit velocity of the acoustic waves through this combination channel.

It is to be noted that this combination channel (AC₁ plus AC₂) can be selectively heated by the activation of laser 50. This mode of operation of the apparatus, therefore, permits laser 50 to be energized or not energized during a glucose monitoring operation. Thus, by activating laser 50 merely to heat the medium within the channel (and not to produce the above-described photoacoustic waves), the temperature of the medium within this combination channel will be raised according to the glucose concentration. Therefore, the magnitude of the output signal from detector 41 also provides useful information since it will be a measure of the glucose concentration diminished by the transit distance to the detector 41, and the factors influencing the acoustic impedance in this combination channel.

Accordingly, this combination channel (AC₁ plus AC₂) may be activated without energizing laser 50 to define a baseline or reference for comparison. This combination channel may also be activated while laser 50 is energized to apply a controlled or measured amount of energy to the medium within this combination channel. Such a two-stage activation of the combination channel thus enables the extraction of information from the monitored site useful in determining the heat influence on the transit time (represented by the frequency of the output signal from detector 41), or on the glucose concentration (represented by the magnitude of the signal output from detector 41), produced by the laser energy absorbed by the targetted glucose within this combination channel.

Information Extractable from Channel AC₁

Acoustic channel AC₃ does not use the photoacoustically generated waves as the transmitter, as in channels AC₁ and AC₂, when individually activated, but rather utilizes acoustic transducer 32 as a transmitter for transmitting acoustic waves through channel AC₃ for reception by detector 42. Therefore, detector 42 would control, via system 60, the frequency of transducer 32 as described above to maintain the number of wavelengths in channel AC₃ as a whole integer. Since the transit distance of this channel is known or can be determined as indicated above, channel AC₃ can also be used for extracting information from the monitored site as to conditions influencing the transit velocity or acoustic impedance of the acoustic waves through that channel. The combination channel AC₁ plus AC₂, however, provides the additional advantage of permitting a two-stage activation of that channel, one stage including heating by the laser, as described above.

Information Extractable from Channel AC₄

Acoustic channel AC₄, defined by transducers 33 and 43, includes the space (air) below the ear lobe. It may therefore be used for providing reference information for determining the precise transit distances of the other three channels as described above, or for determining the influences on the transit times, the transit velocity, or the acoustic impedance imposed by the ear lobe to the acoustic waves transmitted therethrough via the other channels, as described above.

Using the Laser to Produce Acoustic Waves by the Photoacoustic Effect

Acoustic channel AC₁ could be activated by utilizing detector 31 to control the frequency of activation of laser 50 in order to produce a whole integer number of photoacoustic waves in channel AC₁ by the photoacoustic effect as described above. In this case, the frequency of activation of the laser would be influenced by the transit distance (length of channel AC₁) and the transit velocity through channel AC₁. Thus, the frequency of the output signal from detector 31 would be a precise measurement of the transit time of the acoustic wave through channel AC₁. The magnitude of the output signal from detector 31 would be a measure of the amount of laser energy absorbed by the targetted glucose, and thereby a measure of the glucose concentration as diminished by the transit distance and acoustic impedance within channel AC₁.

With respect to the measured transit time as represented by the frequency of the output signal from detector 31, this transit time would depend on the transit distance and the transit velocity of the acoustic wave within channel AC₁.

The transit distance is the length of channel AC₁. This can be determined with extremely high accuracy from the other information extractable from the monitored site via the other channels, as described herein.

The transit velocity is influenced by the physical nature of the medium in channel AC₁ and also by the temperature of the medium in that channel. The medium is the portion of the ear lobe between transducers 31 and 41. It is constituted mainly of tissue and blood containing the targetted glucose whose concentration is to be determined. Information regarding the influence of the targetted glucose, of the tissue, and of the temperature, on the transit velocity of the acoustic waves within channel AC₁ is extractable from the other channels by independently performed tests, such as to enable assessing the magnitude of these influences on the transit velocity, and thereby on the glucose concentration measurements.

Acoustic channel AC₂ could be similarly activated by using detector 41 for controlling laser 50. The frequency and magnitude of this output signal from detector 41 would provide similar information as in channel AC₁ with respect to the factors in influencing the transit velocity of the acoustic waves through that channel.

The combined channel (AC₁ plus AC₂) could also be independently activated, by using transducer 31 as a transmitter and transducer 41 as a detector, and controlling the activation of detector 31 by the output signal from detector 41. Laser 50 could be selectively operated to influence the transit velocity by the selective heating of the combined channel as described above. Such operation would also enable extracting from the monitored site information useful with the other information for determining the medium and/or heat influences on the transit velocity.

Channel AC₃ can be similarly activated for extracting useful information from the monitored site. Thus, by using transducer 42 as a detector for controlling transducer 32 used as a transmitter, the information obtainable from channel AC₃ would depend on the transit distance and transit velocity in that channel. Since the transit distance AC₃ is equal to the sum of the transit distances in the two channels AC₁ and AC₂, and since the transit velocity in channel AC₃ is influenced primarily by the ear lobe tissue and not by the heat generated by the targetted glucose upon activation of the laser 50, information as to these influences is also obtainable from channel AC₃. Such information can be used with the information obtainable when activating the other channels to assess the magnitude of these influences on the transit velocity, and thereby on the determination of the concentration of the glucose in the monitored site.

Acoustic channel AC₄, may also be activated to provide further useful information enabling a precise measurement of the length of channel AC₄ and thereby of the lengths of channel AC₁, AC₂ and AC₃. Channel AC₄ is not affected by the heat generated by target substance TS or by the ear lobe tissue medium, influencing the transit velocity in the above-described channels AC₁-AC₃. Accordingly, the information obtainable from channel AC₄ could also be useful to assess the medium and/or heat influences on the transit velocity, and thereby to enable a more precise measurement of the glucose concentration to be made.

Using the Laser Merely as a Heat Source to Heat the Monitored Site

The apparatus illustrated in FIG. 3 also permits independent measurements to be made using the laser 50 merely as a heat source, rather than as a means for generating photoacoustic waves.

Thus, the FCWC (Frequency-Change by Wavelength-Control) method described above with respect to FIG. 2 (and more particularly described in the above-cited U.S. Pat. No. 6,621,278) can be used for producing a measurement of the concentration of the glucose (or other target substance) according to the amount of heat absorbed from the laser. In the apparatus illustrated in FIG. 3, this would be done in the above-described combination channel AC₁ plus AC₂ by activating laser 50 (e.g., at a measured rate and intensity so as not to damage the tissue) to transmit acoustic waves from transducer 31, acting as a transmitter, to detector 41. As described above, detector 41 would control the frequency of transmitter 31 to produce and maintain a whole integer number of wavelengths in the combination channel (AC₁ plus AC₂) between the transmitter 31 and detector 41.

The frequency of transmitter 31 would, therefore, depend on the transit distance and transit velocity between transmitter 31 and detector 41. The transit distance is known, or determinable as described above. The transit velocity varies with the heat generated by the glucose TS absorbing the laser energy. Since the heat so generated depends on the concentration of the glucose, the difference in frequency of transmitter 31 to maintain the number of wavelength as a whole integer in the combination channel AC₁ plus AC₂ (a) when this channel is activated with the activation of the laser, and (b) when this channel is activated without the activation of the laser, would be a measure of the heat generated within that channel by the glucose, and thereby a measure of the concentration of the glucose in the monitored site.

Such a measurement of the glucose concentration is not dependent on the photoacoustic effect. It therefore can be used alone for determining glucose concentration. Alternatively, it can be used together with above-described method utilizing the photoacoustic effect in order to corroborate the results produced by that measurement, or to extract information from the monitored site useable to increase the reliability and repeatability of the measurements based on the photoacoustic effect.

It will further be seen that another independent measurement of the glucose concentration can be made using the laser merely to heat the monitored site by utilizing the magnitude, rather than the frequency, of the output signal from detector 41. In that case, the magnitude of the output signal would be a measure of the glucose concentration, reduced by the transit distance influence and the acoustic impedance influence to detector 31, as described above. This can be done by activating the combined channel AC₁ plus AC₂ (a) without activating the laser, and then (b) while activating the laser to introduce a measured amount of energy converted to heat by the glucose according to its concentration, and comparing the magnitude of the detector output for both cases. Such an independent measurement of the glucose concentration, although less precise than the measurement based on frequency change, could nevertheless be made to corroborate a frequency-change measurement, and/or to extract from the monitored site information useful in increasing the precision and repeatability of the measurement made by the frequency-change method.

The Embodiment on FIG. 4

FIG. 4 illustrates a modification in the sensor assembly 20 of FIG. 3, wherein transducers 32 and 42, defining acoustic channel AC₃ in FIG. 3, are omitted. In this case, similar information can be obtained as obtained from channel AC₃ in FIG. 2, by using transducer 31 as a transmitter, and transducer 41 as a receiver, to thereby produce the operation described above with respect to the combination channel AC₁ plus AC₂. In such an arrangement, the combination channel could be operated at one time while receiving laser energy, and at another time while not receiving laser energy, so as to provide a base line or reference for measuring the heat influence in the former operation.

In all other respects, the apparatus illustrated in FIG. 4 is constructed, and may be operated, in the same manner as described above with respect to FIG. 3, and therefore to facilitate understanding, the same numerals have been used with respect to corresponding elements.

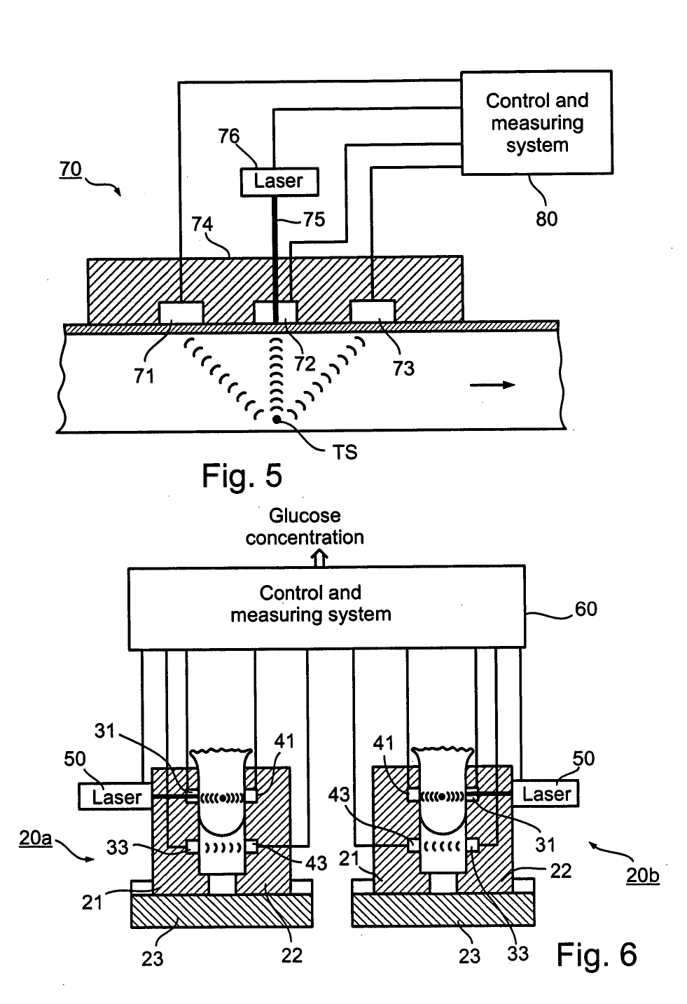

The Embodiment of FIG. 5

FIG. 5 illustrates a further apparatus constructed in accordance with the present invention, similar to that of FIG. 1 in that the monitored site requires access only from one side, thereby permitting a person's wrist, fingertip, or the like, to be used as a monitoring site. The system illustrated FIG. 5 differs from that in FIG. 1 in that the FIG. 5 system provides not a single acoustic channel as in FIG. 1, but rather a plurality of acoustic channels, as described above with respect to FIGS. 3 and 4, to enable various types of information to be extracted from the monitored site during a monitoring operation, such as to increase the reliability and repeatability of the glucose measurement, while reducing the need for frequent recalibration.

Thus, as shown in FIG. 5, the detector assembly, generally designated 70, includes three (or more) piezoelectric transducers 71, 72, 73, mounted in predetermined fixed positions on a mounting plate 74, configured for application to the monitoring site, e.g. a wrist of the person. Center transducer 72 is formed with an opening receiving an optical fiber 75 from a laser 76, such that the laser energy is supplied by pulses through transducer 72 to the target substance TS (e.g., glucose) whose concentration is being monitored. As described above, the absorption of the laser energy by the target substance TS generates heat according to the concentration of the glucose. This heat may be used to generate acoustic waves by the photoacoustic effect, which waves are propagated outwardly in all directions.

In one operation, the three transducers 71-73 may be used as detectors for detecting the so-generated acoustic waves. Thus, a separate acoustic channel is established between the targetted glucose TS and each of the three detectors 71-73. The illustrated apparatus further includes a control and measuring system 80, similar to system 10 (FIGS. 1 and 2) or system 60 (FIGS. 3 and 4) connected to the three detectors 71-73 and to laser 76.

Each of the detectors 71-73, which defines a separate acoustic channel with the targetted glucose TS, may control the laser 76, via control system 80, such that the frequency of the acoustic waves generated in the respective channel is a measure of the transit time of the acoustic wave in that channel. As described above, the transit time is dependent on the transit distance and the transit velocity in the respective channel. Since the locations of the three detectors 71-73 are known relative to each other, the transit distance (e.g., the length of the respective channel) can easily be determined from the data extracted from the three channels of the monitored site. As also described above, the transit velocity in the respective channel is influenced by the nature of the medium (e.g., tissue plus blood including the glucose), and the temperature of the medium. By using three (or more) such channels as illustrated in FIG. 5, such influences can also be determined, or least closely approximated, from the information extracted from the monitored site.

In another operation, one transducer (e.g., 72) could be used as a transmitter of acoustic waves (instead of the targetted glucose by the photoacoustic effect) to the other transducer, and the laser could be used merely to selectively heat the respective acoustic channels. Thus, by selectively activating the two channels via the above-described FCWC method, with and without activating laser 76, information may be obtained useful in determining the influences of the heat and the channel medium on the transit velocity of the acoustic waves at the monitored site.

The Embodiment of FIG. 6

FIG. 1 illustrates an embodiment of the invention wherein a single acoustic channel is created at a single monitoring site, and FIGS. 3-5 illustrate embodiments wherein a plurality of acoustic channels are created at a single monitoring site. FIG. 6 illustrates a further embodiment wherein a plurality of acoustic channels are created at two (or more) monitoring sites.

The two monitoring sites in the embodiment of FIG. 6 are the two ear lobes of the person being tested. Presumably the glucose concentration, and the various influences involved in determining glucose concentration according to the above-described method, are sufficiently similar in the two ear lobes to enable extracting information from one site useful in the determination of the glucose concentration in the other site. If not, one ear lobe can be pre-calibrated with respect to the other.

For purposes of example, FIG. 6 illustrates two sensor assemblies, therein designated 20a, 20b, each constructed as sensor assembly 20 in FIG. 4; therefore, in order to facilitate understanding, corresponding elements are identified by the same reference numerals. Preferably, but not necessarily, both sensor assemblies include a laser 50, to enable operation of the respective sensor assembly according to any one of several possible modes. Thus, the arrangement illustrated in FIG. 6 enables a wide variety of modes of operation to be selected for any particular case in order to extract information from both monitoring sites useful in determining the glucose concentration in the person's blood.

For example, one sensor assembly may be operated according to the above-described “photoacoustic mode”, wherein the laser is used to produce acoustic waves by the photoacoustic effect, while the other sensor assembly is operated according to the above-described “heating mode”, wherein the laser is used merely to heat the monitored site. Another option would be to activate the laser of one sensor assembly in order to generate heat by the selective absorption of the laser energy according to the glucose concentration at the respective site, while the laser in the other sensor assembly is not energized. Thus, the results of the test in the latter site could be used as a baseline or reference for the test results produced in the former site in assessing the influence of the heat absorbed by the glucose in the former site, which absorbs heat in accordance with its concentration.

Many of the other options described above with respect to a single site would also be available in the two-site arrangement of FIG. 6, in order to extract information from the two monitored sites which can be used to either corroborate the test results produced at one site, to increase the accuracy, reliability and repeatability of the test results, or to reduce the need for frequent recalibration of the apparatus.

As indicated above, various monitoring sites could be used. If an ear lobe is used for the monitoring site, the electrode assembly could be constructed as a separate unit for mounting to the ear lobe, whereas the control and display system could be in a separate unit wire-connected to the sensor unit. Another alternative would be to have the control and display unit incorporated in a wristband for mounting on the wrist of the person, and to have wireless communication with the sensor unit mounted on the person's ear lobe.

It will be appreciated that in all of the above-described embodiments, the laser wave length is selected according to the target substance of interest. Thus, if the target substance of interest is blood glucose, the laser wave length would be selected to have a frequency, or combination of frequencies, to generate the maximum level of acoustic waves by the photoacoustic effect in glucose, as described for example in the above-cited US patents. It will be further appreciated that excitation means other than lasers can be used, e.g. microwaves, X-rays, ion-beams, etc, and that other target substances may be monitored, such as other blood constituents, urine constituents, constituents of other biological fluids, and constituents of industrial fluids, solid bodies, etc.

Therefore, while the invention has been described with respect to several preferred embodiments, it is to be expressly understood that these are set forth merely for purposes of example, and that many other variations, modifications and applications of the invention may be made.

1. A method of non-invasively measuring concentration, or change in the concentration, of a target substance within a body, comprising the operations:

2. activating a pulse source to apply to said body a series of pulses of energy highly absorbable by said target substance, as compared to other substances, to heat said body and to generate therein, by the photoacoustic effect, a series of acoustic waves propagated through an acoustic channel in said body at a frequency corresponding to that at which said energy pulses are applied to the body;

3. detecting said acoustic waves to produce an electrical signal having a frequency corresponding to the frequency of said acoustic waves generated by the photoacoustic effect, and thereby to the frequency at which said energy pulses are applied to said body;

4. controlling said pulse source to change the frequency at which said energy pulses are applied to the body, and thereby the frequency of said acoustic waves, such that said detector detects a whole integer number of wavelengths in said acoustic channel irrespective of variations in the target substance concentration within said body;

5. and utilizing a measurement of the frequency, or change in frequency, of said pulses to produce a measurement of the concentration, or change in concentration, of said target substance.

6. The method according to claim 1, wherein the magnitude, or change in magnitude, of said acoustic waves generated by the photoacoustic effect is also utilized in producing a measurement of the concentration, or change in concentration, of said target substance.

7. The method according to claim 1, wherein said pulse source is a laser having a wavelength, or combination of wavelengths, selectively absorbable by said target substance.

8. The method according to claim 1, wherein said target substance is a constituent of a body fluid of a person.

9. The method according to claim 1, wherein said target substance is glucose in the blood of a person.

10. The method according to claim 1, wherein said detector defines with said target substance a first acoustic channel between said target substance and said detector through which said acoustic waves generated by said photoacoustic effect are propagated, and a second acoustic channel between said target substance and a second detector through which said acoustic waves generated by said photoelectric effect are also propagated;

11. and wherein said method further comprises performing said controlling and utilizing operations also with respect to said pulse source and said second detector of said second acoustic channel.

12. The method according to claim 6, wherein said detector in said first acoustic channel is a piezoelectric device which is also operated as a transmitter of acoustic waves through said first and second acoustic channels to said second detector of said second acoustic channel.

13. The method according to claim 6, wherein said method further comprises:

14. providing a piezoelectric acoustic wave generator and a piezoelectric acoustic wave detector defining a third acoustic channel through said body of a length equal to the sum of the lengths of said first and second acoustic channels;

15. and performing said controlling and utilizing operations also with respect to said piezoelectric acoustic wave generator and acoustic wave detector of said third acoustic channel.

16. The method according to claim 6, wherein said method further comprises:

17. providing a further piezoelectric acoustic wave generator and a further piezoelectric acoustic wave detector defining between them a further acoustic channel outside of said body and of a length equal that of said first and second acoustic channels;

18. and performing said controlling and utilizing operations also with respect to said further piezoelectric acoustic wave generator and detector of said further acoustic channel.

19. The method according to claim 1, wherein said method further comprises:

20. providing a piezoelectric acoustic wave transmitter for generating and transmitting acoustic waves through said acoustic channel in said body to said detector;

21. activating said energy source to apply said energy pulses to heat the portion of said body in said acoustic channel according to the concentration of said target substance therein;

22. controlling said piezoelectric acoustic wave transmitter to change its frequency such that said detector detects a whole integer number of wavelengths in said acoustic channel irrespective of variations in the target substance concentration within said body;

23. and utilizing also the frequency, or change in frequency, of the detector output in producing a measurement of concentration, or the change in concentration, of said target substance.

24. The method according to claim 10, wherein the method further comprises utilizing also the measurements of the detector output in producing a measurement of the concentration, or change in concentration, of said target substance.

25. A method of non-invasively measuring the concentration, or change in concentration, of a target substance within a body, comprising:

26. transmitting acoustic waves through an acoustic wave transmission channel in said body to a detector at the opposite end of said acoustic wave transmission channel;

27. applying to said body in said acoustic wave transmission channel, energy highly absorbable by said target substance, as compared to other substances, to heat the portion of said body within said acoustic wave transmission channel according to the concentration of said target substance in said body;

28. detecting said acoustic waves in said transmission channel to output an electrical signal having a frequency corresponding the frequency of said acoustic waves transmitted through said channel by said acoustic wave transmitter;

29. controlling said acoustic wave transmitter to change the frequency thereof such that the detector detects a whole integer number of wavelengths irrespective of variations in the target substance concentration with said body;

30. and utilizing the frequency of said detector output signal to produce a measurement of the concentration, or change in concentration, of said target substance.

31. The method according to claim 12, wherein the magnitude, of said detector output signal is also utilized to produce a measurement of the concentration, or change in concentration, of said target substance.

32. The method according to claim 12, wherein said pulse source is a laser having a wavelength selectively absorbable by said target substance.

33. The method according to claim 12, wherein said target substance is a constituent of a body fluid of a person.

34. The method according to claim 12, wherein said target substance is glucose in the blood of a person.

35. The method according to claim 12, wherein said energy is selectively controlled so as to be supplied in the form of pulses such as to generate in said body, by the photoacoustic effect, a series of acoustic waves also propagated through said channel in the body but at a frequency corresponding to that at which the energy pulses are applied to the body;

36. and wherein said detector is selectively controlled to also detect said photoacoustically generated acoustic waves, to control the energy source supplying said energy pulses to change the frequency of application of the energy pulses to the body, and thereby the frequency of said acoustic waves, such that the detector detects a whole integer number of wavelengths irrespective of variations in the target substance concentration within the body, and to utilize the frequency of said energy pulses in producing a measurement of the concentration, or change in concentration, of the target substance.

37. A method of non-invasively measuring the concentration, or change in concentration, of a target substance within a body, comprising:

38. transmitting acoustic waves through at least two separate acoustic channel in said body;

39. applying to one of said channels energy which is selectively absorbable by the target substance to thereby heat the respective channel according to the concentration of the target substance therein;

40. and measuring the difference in temperature between that in said one channel with respect to that in the other channel, to thereby provide a measure of the concentration, or change in concentration, of the target substance in the body.

41. The method according to claim 18, wherein said two separate channels are in the same monitored site of said body.

42. The method according to claim 18, wherein said two separate channels are in different monitored sites of said body.

43. The method according to claim 18, wherein said difference in temperature is measured by measuring the transit time of an acoustic wave through each of said channels, and subtracting one transit time from the other.

44. The method according to claim 21, wherein the transit time of an acoustic wave is measured in each of said channels by:

45. detecting each acoustic wave at the end of the respective channel;

46. controlling the frequency of transmission of acoustic wave into the respective channel such as to produce a whole integer number of waves in the respective channel;

47. and utilizing the changes in frequency in the respective channel to determine the transit time of the acoustic wave in the respective channel.

48. The method according to claim 22, wherein the difference in the magnitudes of the acoustic waves at the end of the respective channel is also utilized in providing a measurement of the concentration, or change in concentration, of the target substance within the body.

49. The method according claim 18, wherein said energy is applied to one of said channels in the form of pulses to generate said acoustic waves by the photoacoustic effect, as well as to heat the respective channel according to the concentration of the target substance therein.

50. The method according to claim 18, wherein said acoustic waves transmitted through both said channels are generated by piezoelectric devices; and wherein said energy is applied only to one of said channels to heat the respective channel according to the concentration of the target substance therein.

51. The method according to claim 18, wherein said pulse source is a laser having a wavelength selectively absorbable by said target substance.

52. The method according to claim 18, wherein said target substance is a constituent of a body fluid of a person.

53. The method according to claim 18, wherein said target substance is glucose in the blood of a person.

54. Apparatus for non-invasively measuring changes in the concentration, or change in concentration, of a target substance within a body, comprising:

55. a pulse source for applying to said body a series of pulses of energy highly absorbable by said target substance, as compared to other substances, to heat said body and to generate therein, by the photoacoustic effect, a series of acoustic waves propagated through an acoustic channel in said body at a frequency corresponding to that at which said energy pulses are applied to the body;

56. a detector for detecting said acoustic waves to produce an electrical signal having a frequency corresponding to the frequency of said acoustic waves generated by the photoacoustic effect, and thereby to the frequency at which said energy pulses are applied to said body;

57. and a control and measuring system for controlling said pulse source to change the frequency at which said energy pulses are applied to the body, and thereby the frequency of said acoustic waves, such that said detector detects a whole integer number of wavelengths in said acoustic channel irrespective of variations in the target substance concentration within said body; and for utilizing a measurement of the frequency, or change in frequency, of said pulses to produce a measurement of the concentration, or change in concentration, of said target substance.

58. The apparatus according to claim 29, wherein said control and measuring system also utilizes the magnitude, or change in magnitude, of said acoustic waves generated by the photoacoustic effect in producing a measurement of the concentration, or change in concentration, of said target substance.

59. The apparatus according to claim 29, wherein said pulse source is a laser having a wavelength, or combination of wavelengths, selectively absorbable by said target substance.

60. The apparatus according to claim 29, wherein said detector defines with said target substance a first acoustic channel between said target substance and said detector through which said acoustic waves generated by said photoacoustic effect are propagated, and a second acoustic channel between said target substance and a second detector through which said acoustic waves generated by said photoelectric effect are also propagated;

61. and wherein said control and measuring system performs said controlling and utilizing operations also with respect to said pulse source and said second detector of said second acoustic channel.

62. The apparatus according to claim 32, wherein said detector in said first acoustic channel is a piezoelectric device which is also operated as a transmitter of acoustic waves through said first and second acoustic channels to said second detector of said second acoustic channel.

63. The apparatus according to claim 32, wherein said apparatus further comprises:

64. a piezoelectric acoustic wave generator and a piezoelectric acoustic wave detector defining a third acoustic channel through said body of a length equal to the sum of the lengths of said first and second acoustic channels;

65. and wherein said control and measuring system performs said controlling and utilizing operations also with respect to said piezoelectric acoustic wave generator and acoustic wave detector of said third acoustic channel.

66. The apparatus according to claim 32, wherein said apparatus further comprises:

67. a further piezoelectric acoustic wave generator and a further piezoelectric acoustic wave detector defining between them a further acoustic channel outside of said body and of a length equal that of said fist and second acoustic channels;

68. and wherein said control and measuring system performs said controlling and utilizing operations also with respect to said further piezoelectric acoustic wave generator and detector of said further acoustic channel.

69. The apparatus according to claim 29, wherein said apparatus further comprises:

70. a piezoelectric acoustic wave transmitter for generating and transmitting acoustic waves through said acoustic channel in said body to said detector;

71. and wherein said control and measuring system activates said energy source to apply said energy pulses to heat the portion of said body in said acoustic channel according to the concentration of said target substance therein; controls said piezoelectric acoustic wave transmitter to change its frequency such that said detector detects a whole integer number of wavelengths in said acoustic channel irrespective of variations in the target substance concentration within said body; and utilizes also the frequency, or change in frequency, of the detector output in producing a measurement of the concentration, or change in concentration, of said target substance.

72. The apparatus according to claim 36, wherein said control and measuring system utilizes also the magnitude, or change in magnitude, of the detector output in producing a measurement of the concentration, or change in concentration, of said target substance.

73. Apparatus for non-invasively measuring the concentration, or change in concentration, of a target substance within a body, comprising:

74. a transmitter for transmitting acoustic waves through an acoustic wave transmission channel in said body to a detector at the opposite end of said acoustic wave transmission channel;

75. an energy source for applying to said body in said acoustic wave transmission channel energy highly absorbable by said target substance, as compared to other substances, to heat the portion of said body within said acoustic wave transmission channel according to the concentration of said target substance in said body;

76. a detector for detecting said acoustic waves in said transmission channel to output an electrical signal having a frequency corresponding the frequency of said acoustic waves transmitted through said channel by said acoustic wave transmitter;

77. and a control and measuring system for controlling said acoustic wave transmitter to change the frequency thereof such that the detector detects a whole integer number of wavelengths irrespective of variations in the target substance concentration with said body; and for utilizing the frequency, or change in frequency, of said detector output signal to produce a measurement of concentration, or change in concentration, of said target substance.

78. The apparatus according to claim 38, wherein said control and measuring system also utilizes the magnitude of said detector output signal to produce a measurement of said target substance concentration.

79. The apparatus according to claim 38, wherein said pulse source is a laser having a wavelength selectively absorbable by said target substance.

80. The apparatus according to claim 38, wherein said energy source is a pulse source selectively controlled so as to output pulses which generate in said body, by the photoacoustic effect, a series of acoustic waves also propagated through said channel in the body but at a frequency corresponding to that at which the energy pulses are applied to the body;

81. and wherein said control and measuring system selectively controls said detector to also detect said photoacoustically generated acoustic waves; controls said pulse sources to change the frequency of application of the energy pulses to the body, and thereby the frequency of said acoustic waves, such that the detector detects a whole integer number of wavelengths irrespective of variations in the target substance concentration within the body; and utilizes the frequency of said energy pulses in producing a measurement of the target substance concentration.

82. Apparatus for non-invasively measuring the concentration of a target substance within a body, comprising:

83. a transmitter for transmitting acoustic waves through at least two separate acoustic channel in said body;

84. a source of energy for applying to one of said channels energy which is selectively absorbable by the target substance to thereby heat the respective channel according to the concentration of the target substance therein;

85. and a control and measuring system for measuring the difference in temperature between that in said one channel with respect to that in the other channel, to thereby provide a measure of the concentration of the target substance in the body.

86. The apparatus according to claim 42, wherein said control and measuring system measures said difference in temperature by measuring the transit time of an acoustic wave through each of said channels, and subtracting one transit time from the other.

87. The apparatus according to claim 43, wherein said control and measuring system measures the transit time of an acoustic wave in each of said channels by:

88. detecting each acoustic wave at the end of the respective channel;

89. controlling the frequency of transmission of acoustic wave into the respective channel such as to produce a whole integer number of waves in the respective channel;

90. and utilizing the frequency, or change in frequency, in the respective channel to determine the transit time of the acoustic wave in the respective channel.

91. The apparatus according to claim 44, wherein said control and measuring system also utilizes the differences in the magnitudes of the acoustic waves at the end of the respective channel in providing a measurement of the concentration, or change in concentration, of the target substance within the body.

92. The apparatus according claim 42, wherein said source of energy is a pulse source which supplies pulses to one of said channels to generate said acoustic waves by the photoacoustic effect, as well as to heat the respective channel according to the concentration of the target substance therein.

93. The apparatus according to claim 42, wherein said acoustic waves transmitted through both said channels are generated by piezoelectric devices; and wherein said energy is applied only to one of said channels to heat the respective channel according to the concentration of the target substance therein.

94. The apparatus according to claim 42, wherein said pulse source is a laser having a wavelength selectively absorbable by said target substance.
